(19) United States
(12) Patent Application Publication
(10) Pub. No.: US 2024/0231094 A1
(43) Pub. Date: Jul. 11, 2024

(54) MODULAR AUTOMATED OPTOMETRIC MEASUREMENT AND CORRECTION SYSTEM

(71) Applicant: iCRx, Inc., Tucson, AZ (US)

(72) Inventors: Adoum Mahamat, Tucson, AZ (US); Erol Ozgur, Tucson, AZ (US)

(21) Appl. No.: 18/150,990

(22) Filed: Jan. 6, 2023

(51) Int. Cl. G02B 27/01; A61B 3/10

(52) U.S. Cl. CPC G02B 27/0172; A61B 3/1015; A61B 3/0091

(57) ABSTRACT

The present invention features a medical device for testing and correcting visual acuity in a patient. In some embodiments, the medical device may comprise a head-mounted visual acuity measurement system comprising a plurality of miniaturized optical elements and a communication component communicatively coupled to a cloud server, configured to perform visual acuity measurement of an eye of the patient and transmit the measurement to the cloud server. The medical device may further comprise the cloud server comprising a processor capable of executing computer-readable instructions and a memory component comprising computer-readable instructions for performing calculations and data handling on the measurement and transmitting a correction to the measurement system. The measurement system may be further configured to accept the correction from the cloud server and apply the correction to the eye of the patient.

### FIELD OF THE INVENTION

The present invention is directed to a home-based or point-of-care head-mounted visual acuity measurement, correction system that can deliver eye prescriptions within minutes without visiting an eye doctor's office. The invention is a modular automated measurement and correction system that has been divided into front-end and back-end modules.

### BACKGROUND OF THE INVENTION

Eye care is an essential part of the medical universe, and it is also a multi-billion-dollar industry. A significant portion of the world's population requires eye prescriptions for daily use, and this number is growing due to the increased use of advanced visual technologies such as cell phones, television, and laptop or desktop computers.

The World Health Organization anticipates that one-third of the human population will be requiring corrective optics in the next decade. Access to healthcare, both in terms of availability and affordability, will be a serious concern, particularly for low-income communities and developing countries.

Increasing the efficiency of routine optometric exams can help mitigate the difficulties regarding accessing high-quality and affordable eye care. Auto Phoropters and adaptive optics optometric devices are being used to facilitate eye exams, yet the gold standard in the optometry office is still the subjective refractive testing using a phoropter that relies on verbal feedback of the patient for the specialist to determine the correct prescription. Although there are numerous scientific publications, based on extensive human testing, demonstrating the usefulness of objective refraction, the inability to directly observe a corrected binocular image on an autorefractor steers the optometry specialist to resort to subjective refraction for the best interest of the patient.

Recently, the inventors have developed a prototype device that combines an autorefractor and a phoropter into a single unit. The system can measure the refractive errors objectively with high accuracy and apply an instant correction for spherical and cylindrical aberrations using a set of tunable fluidic lenses. It contains integrated visual acuity charts for on-site verification of the correction. The device is operated by an optometrist, who can perform measurements, binocular correction, and fine-tuning of the prescription if necessary. The measurement and correction usually take less than five minutes for each patient. Unlike autorefractors, the prescription is final and no further verification is required. However, this technology also requires the patient to leave their home to get tested.

Clearly, eye care technology needs advancement to address the feasibility of providing people with a solution within minutes within the comfort of their homes or at other convenient locations such as workplaces.

### BRIEF SUMMARY OF THE INVENTION

It is an objective of the present invention to provide devices that allow for a home-based or point-of-care head-mounted visual acuity measurement and correction system that can deliver eye prescriptions within minutes without visiting an eye doctor's office, as specified in the independent claims. Embodiments of the invention are given in the dependent claims. Embodiments of the present invention can be freely combined with each other if they are not mutually exclusive.

The present invention features a medical device for testing and correcting visual acuity in a patient. In some embodiments, the medical device may comprise a head-mounted visual acuity measurement system comprising a plurality of miniaturized optical elements and a communication component communicatively coupled to a cloud server, configured to perform visual acuity measurement of an eye of the patient and transmit the measurement to the cloud server. The medical device may further comprise the cloud server comprising a processor capable of executing computer-readable instructions and a memory component comprising computer-readable instructions for performing calculations and data handling on the measurement and transmitting a correction to the measurement system. The measurement system may be further configured to accept the correction from the cloud server and apply the correction to the eye of the patient.

One of the unique and inventive technical features of the present invention is the portable physical visual acuity measurement component with a connection to a cloud server for calculations. Without wishing to limit the invention to any theory or mechanism, it is believed that the technical feature of the present invention advantageously provides for portable visual acuity measurement and correction with less required processing power within the measurement device itself. None of the presently known prior references or work has the unique inventive technical feature of the present invention.

Furthermore, the technical feature of the present invention contributed to a surprising result. For example, one of ordinary skill in the art would suspect that portable head-mounted systems are only capable of interpreting the refractive errors of the eye. Surprisingly, the present invention differs from these prior arts by its unique ability to also correct the optical aberrations present in the patient's eyes. The patient looking through the invention can readily see a corrected image, made possible by the miniaturized tunable lens system used in the invention. Thus, the inventive technical feature of the presently claimed invention contributed to a surprising result.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 is a schematic of a prior system shown on top and the present invention at the bottom.

FIG. 2 is a rough sketch of the head-mounted system

FIG. 3 shows a simple configuration of the home-based modular automated optometric system

FIG. 4 shows a configuration of the home-based modular automated optometric system including the waveguide. Note the waveguide could be curved.

### DETAILED DESCRIPTION OF THE INVENTION

Following is a list of elements corresponding to a particular element referred to herein:
100 device
200 measurement system
210 dichroic mirror
220 polarizing beam-splitter
230 Shack-Hartmann sensor
240 chip
300 cloud server

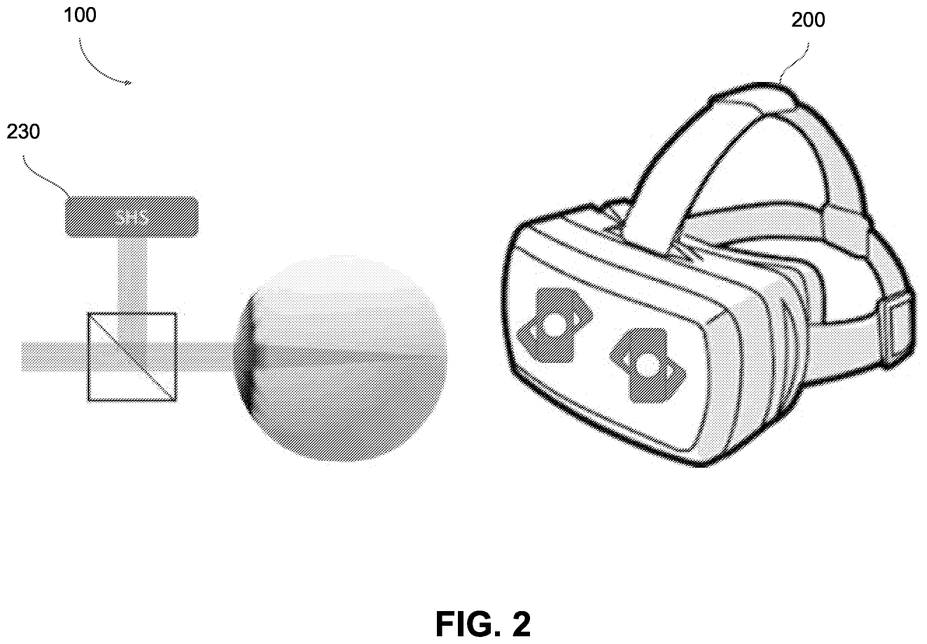

An embodiment of the present invention provides visual acuity measurement and correction in the front-end module, while the calculations and data handling are performed in the back end, which resides in the Cloud. In this embodiment, sensing is partially done in the back end because sensing-related calculations and data analysis take part in the back end. In the invention, the data acquisition and correction are performed by using miniaturized optical and mechanical or electronic elements, including a set of miniaturized tunable optical lenses. Everything fits within a head-mounted display system as shown in FIG. 2. The optical system is simplified and miniaturized to send light to the eye using a waveguide and collect the light scattered back using a miniature optical sensor. The tunable lenses are also miniaturized and placed inside the same structure.

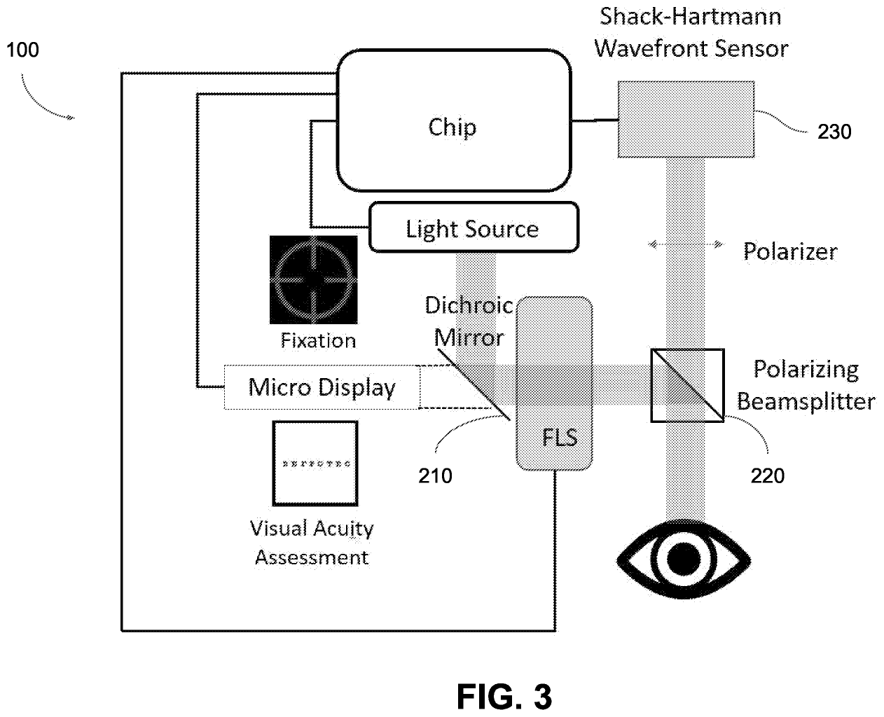

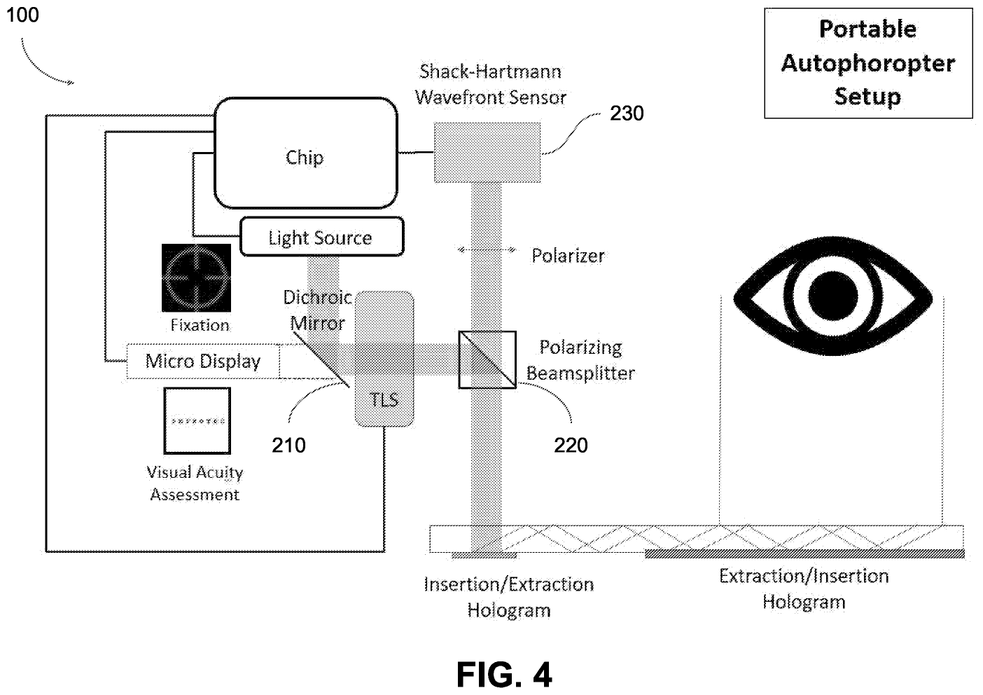

In some embodiments, the front end is miniaturized similarly to FIGS. 3-4. The light is guided towards the eye using a dichroic mirror and a polarizing beam-splitter. In the second embodiment, the light is then inserted in the waveguide using a diffraction/holographic grating and extracted using a second diffraction/holographic grating. The light exiting the waveguide is then sent to the human eye. The light reflected from the eye travels in the opposite direction through the waveguide to the Shack Hartmann Sensor (SHS) for wavefront measurement. Polarization control prevents the cornea reflections to reach the SHS. A tunable lens system corrects the projected image according to the aberration data obtained by the SHS. The projected image is also used to create and maintain patient fixation. All the system is controlled using a chip. The chip transfers the data to be analyzed and receives the output. All the computations take place in the Cloud.

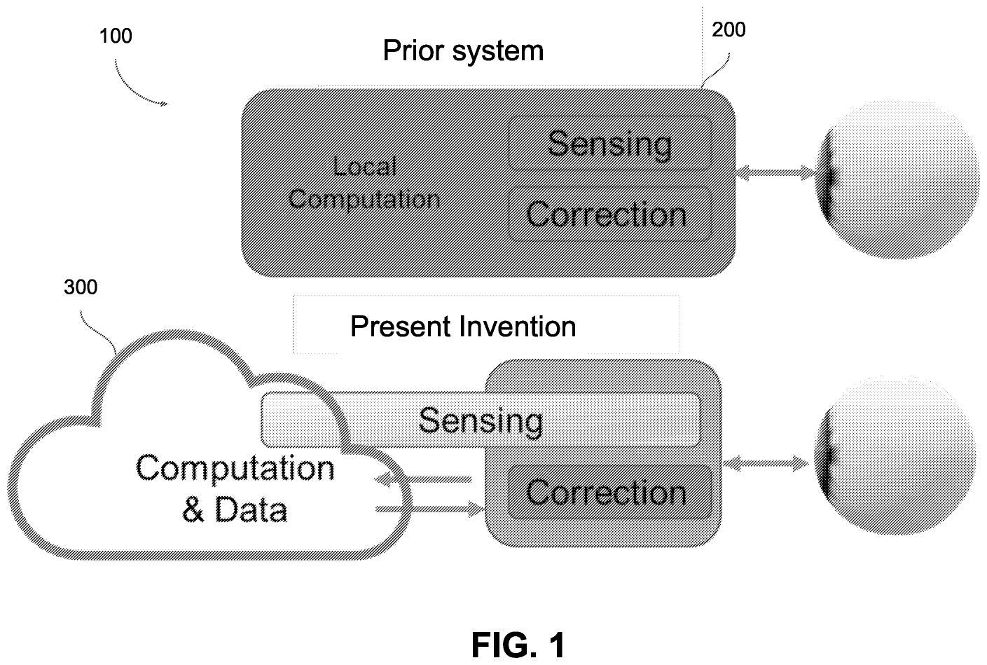

Referring now to FIG. 1, the present invention features a medical device (100) for testing and correcting visual acuity in a patient. In some embodiments, the medical device (100) may comprise a head-mounted visual acuity measurement system (200) comprising a plurality of miniaturized optical elements and a communication component communicatively coupled to a cloud server (300), configured to perform visual acuity measurement of an eye of the patient and transmit the measurement to the cloud server (300). The medical device (100) may further comprise the cloud server (300) comprising a processor capable of executing computer-readable instructions and a memory component comprising computer-readable instructions for performing calculations and data handling on the measurement and transmitting a correction to the measurement system (200). The measurement system (200) may be further configured to accept the correction from the cloud server (300) and apply the correction to the eye of the patient.

In some embodiments, the measurement system (200) may comprise a head-mounted, monocular, or binocular display system. In some embodiments, the memory component may further comprise computer-readable instructions for sensing a visual acuity of the patient. In some embodiments, the measurement system (200) may further comprise a simplified and miniaturized optical system configured to send light to the eye of the patient using a waveguide, and collect the light scattered back using a miniature optical sensor. In some embodiments, the waveguide may be configured to propagate the light back and forth.

In some embodiments, the measurement system (200) may further comprise a dichroic mirror (210) and a polarizing beam-splitter (220). The dichroic mirror (210) and the polarizing beam-splitter (220) may be configured to guide light towards the eye. The waveguide may be configured to accept the light from the dichroic mirror (210) and the polarizing beam-splitter (220) using a first grating, and extract the light using a second grating towards the eye. The first grating may be further configured to couple the light comprising a wavefront reflected from the eye back into the waveguide. The waveguide may be further configured to reflect the light carrying the wavefront in the opposite direction through the waveguide. The first grating may be further configured to direct the light comprising the wavefront to a Shack Hartmann Sensor (SHS) (230) for wavefront measurement.

In some embodiments, the medical device (100) may further comprise a polarization control disposed in line with first grating and the SHS (230), configured to accept the light directed towards the SHS (230) to prevent cornea reflections from reaching the SHS (230). In some embodiments, the measurement system (200) may further comprise a tunable lens system configured to correct a projected image according to aberration data obtained by the SHS (230). The projected image may be additionally used to create and maintain patient fixation. In some embodiments, the medical device (100) may further comprise a chip (240) communicatively coupled to the cloud server (300), configured to transfer the aberration data to the cloud server (300), receive output from the cloud server (300), and apply data compression algorithms to reduce size of the aberration data. In some embodiments, the plurality of miniaturized optical elements may comprise a plurality of miniaturized tunable optical lenses. The first grating may comprise a diffraction grating or a holographic grating. The second grating may comprise a diffraction grating or a holographic grating. The aberration data may comprise spherical aberration data, cylindrical aberration data, or a combination thereof.

In some embodiments, the communication component may comprise an antenna capable of connecting to the cloud server by any form or Internet connection. In some embodiments, the cloud server may comprise a server contained in an external storage component connected to the Internet such that the communication component is able to access the cloud server. The cloud server may further comprise a processor capable of executing computer-readable instructions and a memory component comprising computer-readable instructions able to be executed by the processor.

The computer system can include a desktop computer, a laptop computer, a tablet, or the like and can include digital electronic circuitry, firmware, hardware, memory, a computer storage medium, a computer program, a processor (including a programmed processor), or the like. The computing system may include a desktop computer with a screen and a tower. The tower can store digital images in binary form. The data/images can also be stored in the cloud. The images can also be divided into a matrix of pixels. The pixels can include a digital value of one or more bits, defined by the bit depth. The network or a direct connection interconnects the imaging apparatus and the computer system.

The term “processor” encompasses all kinds of apparatus, devices, and machines for processing data, including by way of example a programmable microprocessor, a computer, a system on a chip, or multiple ones, or combinations, of the foregoing. The apparatus can include special-purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, etc., or any suitable combination of the foregoing. Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user. Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation, a SPARC processor made by Sun Microsystems, an Athlon, Sempron, Phenom, or Opteron processor made by AMD Corporation, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation; the Mac OS X operating system from Apple Computer Corp.; a Unix or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A medical device (100) for testing and correcting visual acuity in a patient, the medical device (100) comprising:
a. a head-mounted visual acuity measurement system (200) comprising a plurality of miniaturized optical elements and a communication component communicatively coupled to a cloud server (300), configured to perform visual acuity measurement of an eye of the patient and transmit the measurement to the cloud server (300); and
b. the cloud server (300) comprising a processor capable of executing computer-readable instructions and a memory component comprising computer-readable instructions for performing calculations and data handling on the measurement and transmitting a correction to the measurement system (200);
wherein the measurement system (200) is further configured to accept the correction from the cloud server (300) and apply the correction to the eye of the patient.

2. The medical device (100) of claim 1, wherein the measurement system (200) comprises a head-mounted, monocular, or binocular display system.

3. The medical device (100) of claim 1, wherein the memory component further comprises computer-readable instructions for sensing a visual acuity of the patient.

4. The medical device (100) of claim 1, wherein the measurement system (200) further comprises a simplified and miniaturized optical system configured to send light to the eye of the patient using a waveguide, and collect the light scattered back using a miniature optical sensor.

5. The medical device (100) of claim 4, wherein the waveguide is configured to propagate the light back and forth.

6. The medical device (100) of claim 5, wherein the measurement system (200) further comprises:
a dichroic mirror (210); and
a polarizing beam-splitter (220);
wherein the dichroic mirror (210) and the polarizing beam-splitter (220) are configured to guide light towards the eye;
wherein the waveguide is configured to accept the light from the dichroic mirror (210) and the polarizing beam-splitter (220) using a first grating, and extract the light using a second grating towards the eye;
wherein the first grating is further configured to couple the light comprising a wavefront reflected from the eye back into the waveguide;
wherein the waveguide is further configured to reflect the light carrying the wavefront in the opposite direction through the waveguide;
wherein the first grating is further configured to direct the light comprising the wavefront to a Shack Hartmann Sensor (SHS) (230) for wavefront measurement.

7. The medical device (100) of claim 6 further comprising a polarization control disposed in line with first grating and the SHS (230), configured to accept the light directed towards the SHS (230) to prevent cornea reflections from reaching the SHS (230).

8. The medical device (100) of claim 6, wherein the measurement system (200) further comprises:
a tunable lens system configured to correct a projected image according to aberration data obtained by the SHS (230);
wherein the projected image is additionally used to create and maintain patient fixation.

9. The medical device (100) of claim 8 further comprising a chip (240) communicatively coupled to the cloud server (300), configured to transfer the aberration data to the cloud server (300), receive output from the cloud server (300), and apply data compression algorithms to reduce size of the aberration data.

10. The medical device (100) of claim 1, wherein the plurality of miniaturized optical elements comprise a plurality of miniaturized tunable optical lenses.

11. The medical device (100) of claim 6, wherein the first grating comprises a diffraction grating or a holographic grating.

12. The medical device (100) of claim 6, wherein the second grating comprises a diffraction grating or a holographic grating.

13. The medical device (100) of claim 8, wherein the aberration data comprises spherical aberration data, cylindrical aberration data, or a combination thereof.
